(19) United States
(12) Patent Application Publication
(10) Pub. No.: US 2005/0244520 A1
(43) Pub. Date: Nov. 3, 2005

(54) Topical menthol, or a related cooling compound, to induce lubrication

(75) Inventors: Ronald J. Thompson, Ft Thomas, KY (US); James M. Thompson, Cincinnati, OH (US); Justin R. Thompson, Cincinnati, OH (US)

Correspondence Address: Donald N. Halgren, 35 Central Street, Manchester, MA, 01944, US

(73) Assignee: 40J's LLC

(21) Appl. No.: 11/174,037

(22) Filed: Jul. 1, 2005

Related U.S. Application Data
(63) Continuation-in-part of application No. 10/731,692, filed on Dec. 9, 2003.
Application No. 10/731,692 is a division of application No. 10/004,091, filed on Oct. 23, 2001, now Pat. No. 6,702,733.
Application No. 10/004,091 is a continuation of application No. 09/520,110, filed on Mar. 7, 2000, now Pat. No. 6,322,493.
Application No. 09/520,110 is a continuation-in-part of application No. 09/469,959, filed on Dec. 21, 1999, now abandoned.
Application No. 09/469,959 is a continuation-in-part of application No. 09/414,250, filed on Oct. 7, 1999, now Pat. No. 6,224,541.
Application No. 09/414,250 is a continuation-in-part of application No. 09/340,227, filed on Jul. 1, 1999, now Pat. No. 6,179,775.

(51) Int. Cl. A61K 35/78

(52) U.S. Cl. 424/739; 424/747; 424/745; 424/742

(57) ABSTRACT

Vaginal Lubrication is an important component of a woman's sexual arousal. The other components are vaginal vasocongestion and the establishment of a clitoral erection. The use of menthol or any related cooling compound, topically applied to the clitoris, evokes a reflex vaginal lubrication, via the clitoral nociceptors, the spinal autonomic nerves, and inducing the reflex release of vasoactivepolypeptides adjacent to the vaginal arterioles. The vaginal lubrication can be an effective therapy for atrophic vaginitis, the most common cause of dyspareunia (pain with intercourse).

### BACKGROUND OF THE INVENTION

### FIELD OF THE INVENTION

This invention relates to arrangements for the stimulation of females and more particularly to topical application of specialized stimulatory medicaments. This application is a continuation-in-part application of co-pending U.S. patent application Ser. No. 10/731,692 filed 9 Dec. 2003 which is a divisional application of U.S. patent application Ser. No. 10/004,091, filed 23 Oct. 2001, now U.S. Pat. No. 6,702,733 issued 9 Mar. 2004, which is a continuation of application Ser. No. 09/520,110, filed 7 Mar. 2000, now U.S. Pat. No. 6,322,493 which is a continuation-in-part of application Ser. No. 09/469,959 filed on 21 Dec. 1999, which is a continuation-in-part of application Ser. No. 09/414,250, filed on 7 Oct. 1999, now U.S. Pat. No. 6,224,541 which is a continuation-in-part of application Ser. No. 09/340,227, filed on 1 Jul. 1999, now U.S. Pat. No. 6,179,775 each of which are incorporated herein by reference in their entirety.

### BACKGROUND OF THE INVENTION

Issued Thompson U.S. Pat. Nos. 6,322,493 and 6,702,733 teach of the topical use of menthol (or any related cooling compound) and L-arginine when applied to the female clitoris to create clitoral vasodilatation and a clitoral erection. The topical menthol has two functions, initial mucous membrane vasodilatation, and secondarily as a lipophilic vehicle (permeability enhancer) to facilitate the transport of L-arginine into the clitoral tissues. The L-arginine substrate excess induces the nitric oxide synthase enzyme to produce nitric oxide and cyclic GMP within the clitoral tissues, especially the corpus cavemosa, to effect and create a clitoral erection. A woman can only achieve an orgasm from stimulation of a completely erect clitoris!

### Clinical Results: Lubrication

In clinical studies of the topical menthol and L-arginine preparations applied directly to the clitoris, a surprising result was a consistent and verified increase in vaginal lubrication. It is not understood how, the topical menthol and L-arginine, when applied to the clitoris, could evoke a reflex vaginal lubrication at a distance of 5 to 10 millimeters away from the site of application.

Women in the clinical study were asked questions regarding both the time to lubricate and the quality of that lubrication. 63 out of 83 women (76%) that completed the study stated in the Pretest either inadequate (or poor) lubrication quality or above average lubrication speeds, or a combination of both. The results of the clinical study show both an increase in the quality of lubrication and a decrease in the time to become lubricated between pre and post tests after using the Menthol/L-arginine product. 66 of the 83 women (79%) reported a decrease in time to achieve lubrication, the average decrease in time being 2.40 minutes from a reported average of 5.90 minutes. Lubrication quality was ranked subjectively by the women on a scale. 57 of the 83 women (69%) reported an increase in the quality of lubrication from the combination. 71 of the 83 women (86%) reported an increase in one or both of the categories and after adjusting for women who reported the max score on pretest (4), 71 of 79 women (90%) actually had increases in one or both category(s). Participants also reported an average of 2.75 uses to achieve a maximum effect. These results exhibit the enhanced lubrication effects of the Menthol, as the average time to lubrication was just 3.5 minutes after initial application directly to the clitoris.

### Vaginal Anatomy: References: Telinde, Bailey

The vagina is a tubular potential space that proximally initiates with the vaginal introitis at the vestibule. The distal vagina terminates at the uterine cervix and the cul-de-sac. After a hysterectomy the terminis of the vagina is the cul-de-sac referred to by gynecologic surgeons as the vaginal cuff, for as the cervix is removed, the cuff of normal vaginal tissue circumferentially dissected off the cervix, is re-approximated to form the vaginal cuff.

The vagina is lined by a stratified squamous—Nonkeratininzed Epithelium—referred to as mucous membrane. The vagina is surrounded by a rich plexes of arterioles and nerves that are web-like and contained within the supporting stroma just beneath the vaginal mucosa. Estrogen is essential to maintain vaginal epithelial and stromal fullness and elasticity of all of the vaginal and vulvar tissues.

### Blood Supply of the Vagina and Sexual Vasocongestion

The vagina receives its blood supply from a branch of the uterine artery, a branch of the internal iliac (hypogastric) artery, and from the pudendal artery. These separate blood supplies merge to form the web-like plexus that surrounds the entire vagina, and insures a reserve capacity of essentially an endless volume of blood. During the non-aroused state the arterioles of this vaginal plexus are contracted. With sexual arousal, vasocongestion is accomplished by the arterioles undergoing vasodilitation. This vasocongestion is mediated by the release of neuropeptides from the nerves that accompany the blood vessels.

Dr. R. J. Levin in the 2002 Arch Sex Behavior article “The Physiology of Sexual arousal in the human female: A recreational and procreational hypothesis,” reports sexual arousal initiates enhanced genital blood flow, leading to the formation of a neurogenic transudate, lubricating the vagina.

The neurogenic transudate that Dr. Levin refers to has two components, the release of a protein polypeptide by the nerve ending coursing with the arteriole, causing the arteriole to dilate and increase its diameter, and therefore its blood volume, and the increase in the arteriolar permeability to allow the vaginal lubricating transudate.

The nerves of the vagina originate from the S₂, S₃, and S₄ nerve roots of the spinal cord, the autonomic paravertebral nerves (S₂-S₄), and even the terminus of the autonomic vagus nerves. The vagina has no sensory nerve endings within the mucosa, as does the clitoris. Therefore, pain or pleasure cannot be perceived from the vaginal mucosa. Motor nerves from S₂-S₄ nerve roots supply the smooth muscles that support and define the vagina. The autonomic paravertebral, and to a lesser degree, the autonomic vagus nerves are responsible for the release of the neurogenic protein polypeptides that effect vaginal vasocongestion and vaginal lubrication. Nerves course with blood vessels in neurovascular bundles and are given names of the partner blood vessel.

### Menthol/Clitoral Anatomy

Eccles reports on the permeation enhancing actions of menthol, to function as a vehicle to topically deliver pharmaceutical compounds across a tissue type, as being dependent upon the tissue type. Epidermis is relatively non-absorptive, mucous membrane is absorptive, and the conjunctiva of the eye is very absorptive. Dr. Eccles reports a concentration of menthol of 10 times for epidermis, one time for mucous membrane, and 0.1 times for conjunctiva to achieve the desired tissue permeability. This is explained by the different tissue types described in Bailey's Histology Text, the standard teaching Histology/Microscopic Anatomy for Medical Students.

Epidermis (skin/hairy skin) is a stratified squamous keratinized tissue. The conified layer of the epidermis is composed of “soft keratin” (“hard keratin” is found in nails and hair). This “soft keratin” presents a relatively impenetrable barrier to all topical pharmaceuticals unless they are lipophilic and maintained in a reservoir in contact with the epidermis like transdermal adhesive patch technology. Topical menthol (BenGay®) requires a strength of 10% menthol for effect on hairy skin.

Mucous membranes (mucosal) lines all of the cavities and canals of the body which connect to the exterior, vagina/vulvar/mouth/respiratory system/anus/rectum. Mucous membrane does not contain “soft keratin.” The mucous membrane is stratified squamous tissue containing variable amounts of mucous glands depending on the tissue type (vestibule>mouth >respiratory tissue). Mucous membrane is 10 times more absorptive then epidermis due to lack of the conified barrier. Bailey describes the “vestibule” (external female anogenital vulvas) as containing multiple small mucous glands. The glandulae vestibulares minores are placed chiefly near the clitoris and opening of the urethra. Bailey also describes the clitoris as consisting mainly of erectile tissue similar to that of the corpus cavernosa of the penis. It is covered with a thin stratified squamous epithelium, underneath which is a papillated stroma rich in blood vessels and containing numerous sensory nerve fibers with highly specialized termination, such as Meissner's corpuscles and Pacinian corpuscles.

Meissners's corpuscles are encapsulated demylinated nerve endings that respond to tactile stimuli (fme touch). Pacinian corpuscles are large multiple layered onion-like capsules of connective tissue surrounding a single demylinated nerve stimulated by pressure of a heavy nature.

Nociceptors are defined by Drs. McCleskey and Gold in “Ion Channels of Nociception,” published in the 1999 Annual Review of Physiology, as transducing noxious stimuli into depolarizations that trigger action potentials, conducting the action potentials from the peripheral sensory site to the synapse in the central nervous system, and converting the action potentials into neurotransmitter release at presynaptic terminal.

### Glossary of Terms

1. Nociceptors—sensory nerve endings—responsive to pressure cold/menthol/heat and certain chemicals like capsiacin.
2. Bulbocavemosa Reflex—(also called clitoroanal refles or CAR)—a spinal reflex arc where pinching of the clitoris causes a reflex involuntary contracture of the anal sphinctor muscle—reassures that nerve roots S₂, S₃, and S₄ are without neurologic pathology.
3. Neurogenic Transdurate—A mechanism where protein polypeptides are released by nerve stimulation. These polypeptides cause an increase in local blood flow and capillary permeability to allow the fluid component of the circulating blood to leak out of the lumen of the capillary. This fluid component of the blood (plasma) leaks from the dermis, the deepest layer of the mucous membrane, through the epidermis, a non-keratinized stratified squamous, and into the lumen of the vagina. This natural vaginal lubrication is a transudate, for it transits the capillary wall and both layers of the mucous membrane into the lumen of the vagina. This is the initial physiological response in a woman's sexual arousal process.
4. Vasoactive Neuropeptides—(also named VIP—Vasoactive Intestinal Peptides in the 1980s and 1990s)—the actual specific polypeptides that effect the capillary walls with a vasodilitory (causing vaginal blood engorgement) effect, and increased capillary permeability, to allow the plasma transudate for vaginal lubrication.
5. TRP—(Transient Receptor Potential)—a nociceptor classification where the response to a specific stimulating agent is named after that agent.
6. TRPM8—(Transient Receptor Potential—Menthol 8)—the specific nociceptor (receptor) for cold and menthol.
7. Pelvic Nerve Stimulation—an in vivo research tool that is used to induce vaginal lubrication by electrical stimulation of the autonomic nerves that supply the vagina.

### Importance of and Causes of Decreased or Absent Vaginal Lubrication

A woman's sexual arousal involves three recognizable physiological responses, vaginal vasocongestion, vaginal lubrication, and clitoral erection. Dr. Levin in his article, “VIP, vagina, clitoral, and periurethral glans—an update on human female genital arousal,” from the 1991 Journal of Experimental Clinical Endocrinology states, “The vaginal lubrication created (by plasma transudation) allows painless penile penetration and coital movements.”

In the 1999 CME Levin article, “Female Sexual Dysfunction: Incidence, Pathophysiology, evaluation, and treatment options,” published in Urology, Drs. Berman, Berman, and Goldstein define sexual arousal disorder as persistent or recurring inability to attain, or maintain sufficient sexual excitement, causing personal distress—Disorders of arousal include, but are not limited to, lack or diminished vaginal lubrication.

Two well recognized causes of decreased or absent ability to adequately lubricate are normal aging and contraceptive use. Normal aging of a woman entails the slow gradual decline in estrogen production from the ovaries and culminates with the menopause, where the ovaries cease estrogen production. The ten or more years preceding the menopause are referred to as the perimenopause. Patients report noticing inadequate vaginal lubrication with intercourse usually in their early to mid thirties, the onset of the perimenopause. In a report published in Biology of Reproduction 2004, Dr. Ting et al, state in their article titled, “Estrogen regulates vaginal sensory and autonomic nerve density in the rat,” that vaginal dysfunction during menopause is generally assumed to occur because of diminished estrogen—mediated trophic support of vaginal target cells. Dr. Ting also reports as a conclusion that, “These findings indicate that some aspects of vaginal dysfunction during menopause may be attributable to changes in innervation.”

An article entitled “Effects of ovariectomy and estrogen replacement on basal and pelvic nerve stimulated vaginal lubrication in an animal model,” published in the 2003 Journal of Sex and Marital Therapy by Dr. Min, of Dr. Goldstein's research group, concludes “Estrogen replacement normalized lubrication values and tissue wet weight to control levels. In conclusion, vaginal tissue integrity and lubrication are diminished by ovariectomy (removal of ovary) and are normalized by estrogen replacement.”

In addition to the normal aging related decreased vaginal lubrication as a cause of female sexual dysfunction, contraceptive medications in young patients cause inadequate or absent vaginal lubrication. This is easy to understand because all contraceptive medications involve a hypoestrogenic state. Combination estrogen/progesterone oral control pills or transdermal patches deliver a small daily dose of estrogen to suppress all ovarian functions. The small daily dose of estrogen prevents the development of a follicle containing the ova, hence the contraception, but also this small dose of estrogen suppresses the normal levels of estrogen production from the ovaries. The dose of daily estrogen needed to suppress all ovarian function is a small fraction, one third to one half, of the amount of estrogen normally produced from the ovaries if the patient were not taking the contraceptive medication. In the 2004 journal, Contraception, an article entitled, “Sexual behavior of women taking low dose oral contraceptive containing 15 microgram ethinyl estradiol/60microgram gestodene,” Dr. Caruso concludes “Pharmacologically induced symptoms (of the low dose estrogen pill) . . . act on sexual aspects of the subjects, decreasing sex desire and vaginal lubrication.”

Therefore, both conditions associated with decreased or absent ability to adequately sexually arouse and produce vaginal lubrication adequate for intercourse, involve hypoestragenic states, menopause (and Perimenopause) and contraceptive use. This can be understood by the conclusion of Dr. Gorodeski, “Therefore, the hypoestrogenism—related decrease in R (TJ) (intracellular tight junctions) and the hypoestrogenism—and aging-realated increase in R (LIS) (resistance of the lateral intercellular space) could be the cellular mechanisms of decreased permeability that lead to decreased fluid transport and decreased lubrication of the lower genital tract in older postmenopause women.” Dr. Gorodeski's article, “Aging and estrogen effects on transcervical-transvaginal epithelial permeability,” was published in the January 2005 issue of Journal of Clinical Endocrinology and Metabolism. This most recent medical reference establishes the mechanism of the hypoestragenic condition and the decreased or absent vaginal lubrication of arousal disorders of Female Sexual Dysfunction.

### BRIEF DESCRIPTION OF THE DRAWINGS

Referring to the drawings as a labeled description of the female anatomy as utilized with the present invention as follows:

FIG. 1 represents a portion of the nervous system associated with the female anatomy showing the vaginal introitis, the clitoris and the afferent and efferent nerves;

FIG. 2 displays the vaginal arterioles;

FIG. 3A represents an enlargement view of an arteriole relative to vaso active neuropeptides stored in terminus of efferent nerve;

FIG. 3B represents efferent autonomic nerves-terminus and arterioles contracted;

FIG. 3C represents an enlargement of the release of vasoactive neuroptides during sexual arousal—reflex vaginal lubrication, with an arteriole dilated thus having increased permeability;

FIG. 3D displays a plasma transudate from arterioles, an arteriole dilated for increased permeability from neuropeptides;

FIG. 4A displays a view of a normal non-aroused state in a sectional view of a vaginal lumen showing the mucous membrane, arterioles contracted with a thick wall, efferent autonomic nerves-terminus and vaso neuropeptides stored in nerve terminus; and

FIG. 4B displays the sexual arousal reflex vaginal lubrication, with a vaginal lubrication-plasma transudate, dilated arteriole having thinned alls to allow plasma transudate movement, efferent autonomic nerves-terminus, and vasoactive neuropeptides released by nerve endings.

### DESCRIPTION OF THE PRESENT INVENTION

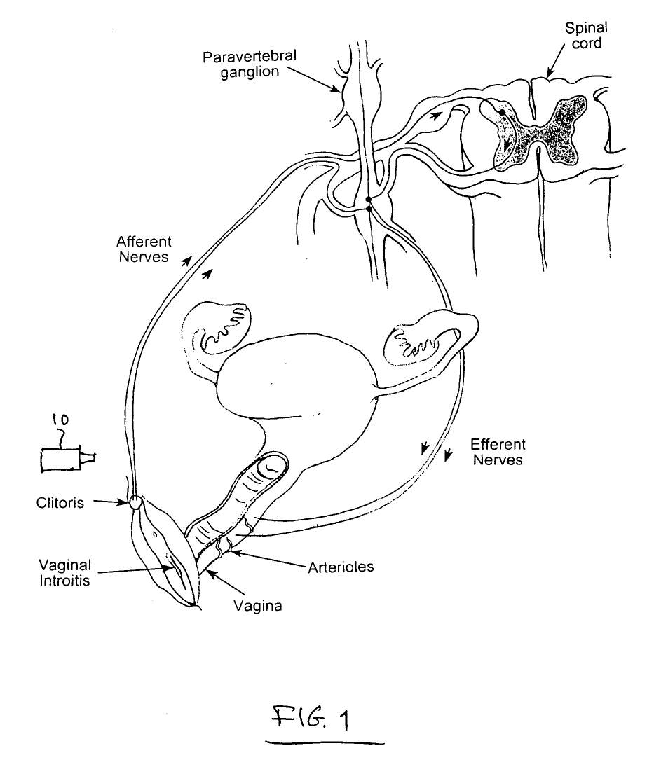

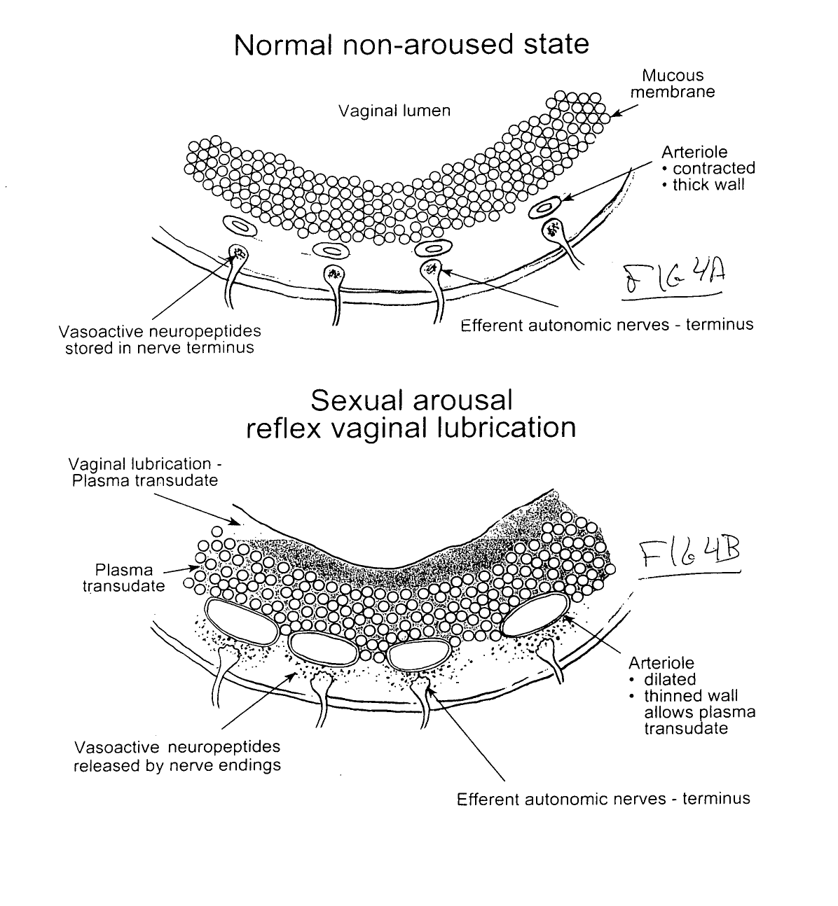

The present invention may be described as the topical application of menthol, a menthol derivative, or menthol analog, or any other related compound from a reservoir 10, represented in FIG. 1, to the female clitoris, to effect a reflex vaginal lubrication represented in FIG. 4B, without the use of topical or systemic estrogen. Building upon the knowledge of the previously cited medial reports, the use of the topical or systemic estrogen, to estrogenize the vaginal mucosa would enhance the ability of menthol or related cooling compound typically applied to the clitoris to produce an even more pronounced reflex vaginal lubrication. The volume and concentration of the menthol to be topically applied to the clitoris must be enough to create the reflex vaginal lubrication adequate for intercourse but less than would cause topical “burning” when applied.

Dr. Brandell et al. in the 2004 journal Neuron Report, entitled “Noxius cold ion channel TRPAI is activated by pungent compounds and brady kinin,” stated “The burning and cooling perception of capsaicin and menthol demonstrate that these ion channels mediate thermosensation. These data demonstrate that the TRPAI activation elicits a painful sensation and provides a potential molecular model for why noxious cold can paradoxically be perceived as burning pain.”

The concentration of 0.5% menthol seems to be the upper limit of tolerability of the topical application of 1-2 cc of menthol to the clitoris. Even this produces reports of “burning” in 10-25% of women. The concentration of 0.01% menthol does not produce “burning” but also does not however, produce the reflex vaginal lubrication when applied topically to the female clitoris.

The invention thus comprises a reflex vaginal lubrication methodology arrangement comprising the steps of a topical manual application of a cooling compound preferably from a reservoir, to the female clitoris, wherein a cooling agent in said compound in said reservoir includes an agent selected from the following group, the agent “critically” having a concentration of less than 0.5% and a concentration greater than 0.01%; wherein said cooling agent includes Menthol; wherein said cooling agent includes peppermint oil; wherein said cooling agent includes cornmint oil; wherein said cooling agent includes Eucalypus oil; wherein said cooling agent includes Citronella oil; wherein said cooling agent includes Camphor oil; wherein said cooling agent includes Cinnamon oil; wherein said cooling agent essentially comprises Menthol; wherein said cooling agent essentially comprises peppermint oil; wherein said cooling agent essentially comprises commint oil; wherein said cooling agent essentially comprises Eucalyptus oil; wherein said cooling agent essentially comprises Citronella oil; wherein said cooling agent essentially comprises Camphor oil; wherein said cooling agent essentially comprises Cinnamon oil; wherein said cooling agent includes a menthol analog or derivative with cooling properties; and

### Menthol Analogs and Derivatives

(+)-neo-Menthol
Menthone
(+)-iso-Menthone
Menthyl acetate
Menthyl isovalerate
(−)-Menthyl lactate
para-menth-1-en-3ol
Piperitone
(−)-Menthol ethylene glycol carbonate
(−)-Menthol 1-and 2-propylene glycol
carbonate
(−)-Menthone 1,2-glycerol ketal
(+)-Menthone 1,2-glycerol ketal
mono-Menthyl succinate

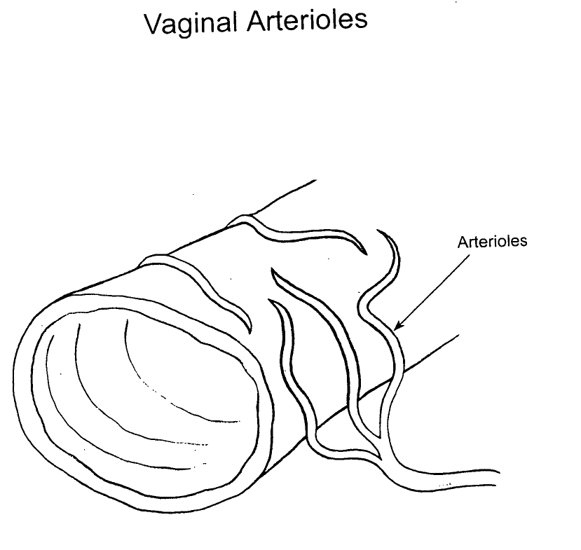

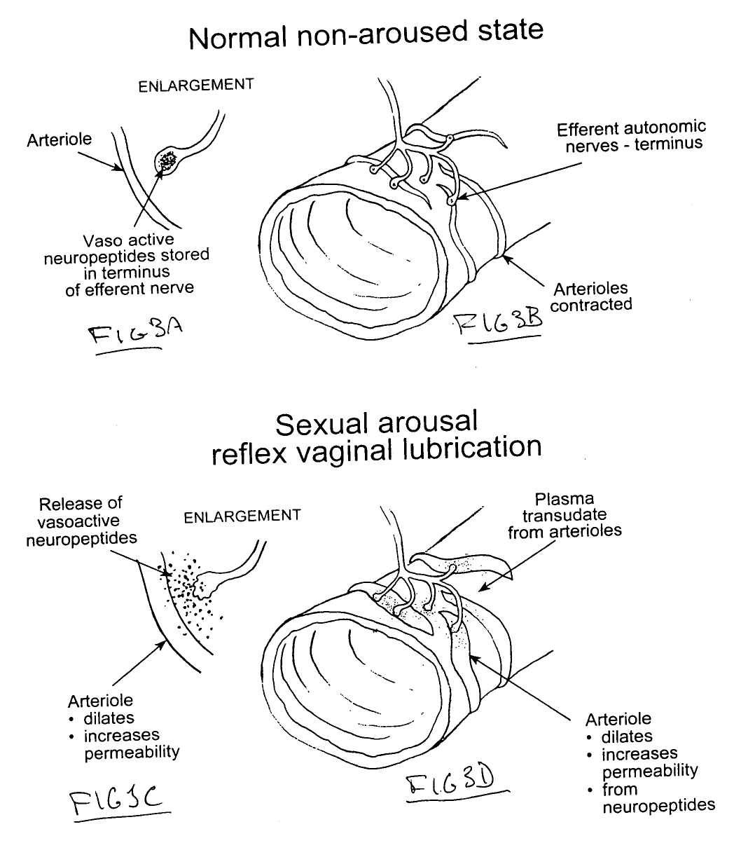

The invention may also include: the cooling agent in a critically controlled concentration, which is applied topically to clitoral nociceptors for stimulation thereof; stimulation of the clitoral nociceptors by the topical manual application of a delivery compound containing menthol or a related cooling compound; a method to induce the release of vasoactive polypeptides, as represented in various stages through FIGS. 2, 3A, 3B, 3C, 3D, 4A and 4B, that directly effect the vaginal arteriole plexes through stimulation of the clitoral nociceptors by the application of a compound containing menthol or a related cooling compound; a method to dilate the vaginal arteriole plexes, through release of vasoactive polypeptides, through stimulation of the clitoral nociceptors by the application of a compound containing menthol or a related cooling compound; a method to increase the permeability of the vaginal arteriole plexes, through release of vasoactive polypeptides, through stimulation of the clitoral nociceptors by the application of a compound containing menthol or a related cooling compound; a method to increase the volume of serum transudate released by the vaginal arteriole plexes through the stimulation of the clitoral nociceptors by the application of a compound containing menthol or a related cooling compound; a method to increase the volume of vaginal transudate (vaginal moisture/lubrication) through the stimulation of the clitoral nociceptors by the application of a compound containing menthol or a related cooling compound; a method to induce vaginal lubrication through the application of the delivery compound containing menthol or a related cooling compound to the clitoral nociceptors eliminating the concurrent use of topical or systemic estrogen.; a method to treat vaginal moisture insufficiency without using an additional intra-vaginal moisturizer, through stimulation of the clitoral nociceptors by the application of a topical compound containing menthol or a related cooling compound; a method to provide natural vaginal lubrication adequate for intercourse, without the use of additional intra-vaginal lubricants through stimulation of the clitoral nociceptors from the application of a topical compound containing menthol or a related cooling compound; a method to provide natural vaginal lubrication adequate for intercourse, in conjunction with the use of an unlubricated male condom, through stimulation of the clitoral nociceptors from the application of a topical compound containing menthol or a related cooling compound; a method to treat sexual arousal disorder, a subset of Female Sexual Dysfunction by providing natural vaginal lubrication adequate for intercourse through stimulation of the clitoral nociceptors by the application of a topical compound containing menthol or a related cooling compound; a method to treat inadequate lubrication, atrophic vulvitis, and dysparunia, a defined subset of Female Sexual Dysfunction by providing natural vaginal lubrication adequate for intercourse through stimulation of the clitoral nociceptors by the application of a topical compound containing menthol or a related cooling compound.

1. A reflex vaginal lubrication arrangement comprising the steps of a topical manual application of a cooling compound from a reservoir, to the female clitoris, wherein a cooling agent in said compound in said reservoir includes an agent selected from the following group, said agent having a concentration of less than 0.5% and greater than 0.01%;
a) wherein said cooling agent includes Menthol;
b) wherein said cooling agent includes peppermint oil;
c) wherein said cooling agent includes cornmint oil;
d) wherein said cooling agent includes Eucalypus oil;
e) wherein said cooling agent includes Citronella oil;
f) wherein said cooling agent includes Camphor oil;
g) wherein said cooling agent includes Cinnamon oil;
h) wherein said cooling agent essentially comprises Menthol;
i) wherein said cooling agent essentially comprises peppermint oil;
j) wherein said cooling agent essentially comprises commint oil;
k) wherein said cooling agent essentially comprises Eucalyptus oil;
l) wherein said cooling agent essentially comprises Citronella oil;
m) wherein said cooling agent essentially comprises Camphor oil;
n) wherein said cooling agent essentially comprises Cinnamon oil;
o) wherein said cooling agent includes a menthol analog or derivative with cooling properties;
Menthol Analogs and Derivatives
(+)-neo-Menthol
Menthone
(+)-iso-Menthone
Menthyl acetate
Menthyl isovalerate
(−)-Menthyl lactate
para-menth-1-en-3ol
Piperitone
(−)-Menthol ethylene glycol carbonate
(−)-Menthol 1-and 2-propylene glycol carbonate
(−)-Menthone 1,2-glycerol ketal
(+)-Menthone 1,2-glycerol ketal
mono-Menthyl succinate

2. The method as recited in claim 1, wherein said cooling agent is applied topically to clitoral nociceptors for stimulation thereof.

3. The method as recited in claim 1, to stimulate the clitoral nociceptors by the topical manual application of a delivery compound containing menthol or a related cooling compound.

4. A method to induce the release of vasoactive polypeptides that directly effect the vaginal arteriole plexes through stimulation of the clitoral nociceptors by the application of a compound containing menthol or a related cooling compound recited in claim 1.

5. A method to dilate the vaginal arteriole plexes, through release of vasoactive polypeptides, through stimulation of the clitoral nociceptors by the application of a compound containing menthol or a related cooling compound recited in claim 1.

6. A method to increase the permeability of the vaginal arteriole plexes, through release of vasoactive polypeptides, through stimulation of the clitoral nociceptors by the application of a compound containing menthol or a related cooling compound recited in claim 1.

7. A method to increase the volume of serum transudate released by the vaginal arteriole plexes through the stimulation of the clitoral nociceptors by the application of a compound containing menthol or a related cooling compound recited in claim 1.

8. A method to increase the volume of vaginal transudate (vaginal moisture/lubrication) through the stimulation of the clitoral nociceptors by the application of a compound containing menthol or a related cooling compound recited in claim 1.

9. A method to induce vaginal lubrication through the application of said delivery compound containing menthol or a related cooling compound recited in claim 1 to the clitoral nociceptors eliminating the concurrent use of topical or systemic estrogen.

10. A method to treat vaginal moisture insufficiency without using an additional intra-vaginal moisturizer, through stimulation of the clitoral nociceptors by the application of a topical compound containing menthol or a related cooling compound recited in claim 1.

11. A method to provide natural vaginal lubrication adequate for intercourse, without the use of additional intra-vaginal lubricants through stimulation of the clitoral nociceptors from the application of a topical compound containing menthol or a related cooling compound recited in claim 1.

12. A method to provide natural vaginal lubrication adequate for intercourse, in conjunction with the use of an unlubricated male condom, through stimulation of the clitoral nociceptors from the application of a topical compound containing menthol or a related cooling compound recited in claim 1.

13. A method to treat sexual arousal disorder, a subset of Female Sexual Dysfunction by providing natural vaginal lubrication adequate for intercourse through stimulation of the clitoral nociceptors by the application of a topical compound containing menthol or a related cooling compound recited in claim 1.

14. A method to treat inadequate lubrication, atrophic vulvitis, and dysparunia, a defined subset of Female Sexual Dysfunction by providing natural vaginal lubrication adequate for intercourse through stimulation of the clitoral nociceptors by the application of a topical compound containing menthol or a related cooling compound recited in claim 1.
